(19) United States
(12) Patent Application Publication
(10) Pub. No.: US 2021/0408843 A1
(43) Pub. Date: Dec. 30, 2021

(54) CLOSED LOOP POWER REGULATION FOR A TRANSCUTANEOUS ENERGY SYSTEM

(71) Applicant: Medtronic, Inc., Minneapolis, MN (US)

(72) Inventors: Jonathan P. Roberts, Coon Rapids, MN (US); Joel B. Artmann, Elk River, MN (US); David J. Peichel, Minneapolis, MN (US); Jacob A. Roe, North St. Paul, MN (US); Michael Kemmerer, Victoria, MN (US)

(21) Appl. No.: 17/361,669

(22) Filed: Jun. 29, 2021

Related U.S. Application Data
(60) Provisional application No. 63/046,051, filed on Jun. 30, 2020.

(51) Int. Cl. H02J 50/80; H02J 50/10

(52) U.S. Cl. CPC H02J 50/80; A61M 60/90; H02J 50/10

(57) ABSTRACT

A controller implantable within the body of a patient as part of a left ventricular assist device (LVAD) system and a method therefore are provided. According to one aspect, the controller includes processing circuitry configured to determine a voltage difference by determining a difference between a first voltage obtained from an internal coil of the controller and a target voltage, and is further configured to encode the voltage difference to produce an encoded voltage difference message. The internal coil is configured to transmit the encoded voltage difference message to a power transmitter to enable closed loop control of power transfer from the power transmitter to the controller to drive the voltage difference toward zero.

### CROSS-REFERENCE TO RELATED APPLICATION

This application claims the benefit of U.S. application Ser. No. 63/046,051, filed Jun. 30, 2020.

### FIELD

The present technology is generally related to implanted medical devices such as a left ventricular assist device (LVAD), and more particularly to closed loop power regulation for a transcutaneous energy transfer system (TETS).

### BACKGROUND

Referring to FIG. 1, an implanted LVAD system 10 has internal components (in the body of the patient) and external components. The LVAD system 10 may typically include an LVAD pump 12 an implanted controller (i-controller) 14 having an internal battery 16, an implanted internal transcutaneous energy transfer system (TETS) coil (i-coil) 18, an external TETS coil (e-coil) 20 and an external power transmitter 21 with a detachable battery 24. In operation, power is supplied from the external power transmitter 21 to the i-controller 14 via mutual coupling of the coils 18 and 20, in order to charge the internal battery 16 of the i-controller 14 and to power the LVAD pump 12. The coils 18 and 20 transfer power by mutual induction of electromagnetic energy over the air and through the body. The power supplied by the external power transmitter 21 may come from the detachable battery 24 or from a wall outlet, for example.

### SUMMARY

The techniques of this disclosure generally relate to closed loop power regulation for a transcutaneous energy transfer system (TETS).

According to one aspect, the present disclosure provides a controller implantable within the body of a patient as part of a left ventricular assist device (LVAD) system or other implanted medical device system. The controller includes processing circuitry configured to determine a voltage difference by determining a difference between a first voltage obtained from a rectified supply voltage of the controller and a target voltage, and to encode the voltage difference to produce an encoded voltage difference message. The controller includes the internal coil configured to transmit the encoded voltage difference message to a power transmitter to enable closed loop control of power transfer from the power transmitter to the controller to drive the voltage difference toward zero.

According to this aspect, in some embodiments, the encoding includes combining the voltage difference with secondary performance information. In some embodiments, the voltage difference and the secondary performance information are encoded using binary phase shift keying (BPSK). In some embodiments, BPSK is preferred over more complex encoding schemes. In some embodiments, the processing circuitry is further configured to synchronize transmission of the encoded voltage difference message is synchronized to an alternating current (AC) applied to an external coil electromagnetically coupled to the internal coil. In some embodiments, the encoded voltage difference message modulates a load on the internal coil.

According to another aspect, a method implemented in a controller implantable within the body of a patient as part of an implanted medical device includes determining a voltage difference by determining a difference between a first voltage obtained from a rectified supply voltage of the controller and a target voltage. The method also includes encoding the voltage difference to produce an encoded voltage difference message. The method further includes transmitting the encoded voltage difference message to a power transmitter via the internal coil to enable closed loop control of power transfer from the power transmitter to the controller to drive the voltage difference toward zero.

According to this aspect, in some embodiments, the encoding includes combining the voltage difference with secondary performance information. In some embodiments, the voltage difference and the secondary performance information are encoded using binary phase shift keying (BPSK). In some embodiments, the transmission of the encoded voltage difference message is synchronized to an alternating current (AC) applied to an external coil electromagnetically coupled to the internal coil. In some embodiments, the encoded voltage difference message modulates a load on the internal coil.

According to yet another aspect, a power transmitter configured to transmit power to an implanted medical device is provided. The power transmitter includes processing circuitry configured to decode an encoded voltage difference message received from an internal controller of the implanted medical device, the voltage difference message having a voltage difference, the voltage difference being proportional to a difference between a first voltage obtained from a rectified supply voltage of the implanted medical device and a target voltage. The processing circuitry is further configured to, responsive to the voltage difference, cause a change in current applied to an external coil position-able to couple power to the internal coil, the change in current determined to drive the voltage difference toward zero.

According to this aspect, in some embodiments, the causing of a change in current applied to the external coil includes adjusting a duty cycle of pulse width modulation of the current applied to the external coil based on a difference between a current in the external coil and an adjustment signal based on the voltage difference. In some embodiments, the current determined in response to the voltage difference is generated by a power proportional integral derivative (PID) controller. In some embodiments, the processing circuitry further includes a digital filter to extract the encoded voltage difference message from a current signal sensed on the external coil. In some embodiments, the processing circuitry is further configured to extract secondary performance information from the voltage difference message.

According to another aspect, a method in a power transmitter configured to transmit power to an implanted medical device is provided. The method includes decoding an encoded voltage difference message received from an internal controller of the implanted medical device, the voltage difference message having a voltage difference, the voltage difference being proportional to a difference between a first voltage obtained from an internal coil of the implanted medical device and a target voltage. The method also includes, responsive to the voltage difference, causing a change in current applied to an external coil position-able to couple power to the internal coil, the change in current determined to drive the voltage difference toward zero.

According to this aspect, in some embodiments, the causing of a change in current applied to the external coil includes adjusting a duty cycle of pulse width modulation of the current applied to the external coil based on a difference between a current in the external coil and an adjustment signal based on the voltage difference. In some embodiments, the adjustment signal based on the voltage difference is generated by a power proportional integral derivative (PID) controller. In some embodiments, the processing circuitry further includes a digital filter to extract the encoded voltage difference message from a current signal sensed on the external coil. In some embodiments, the processing circuitry is further configured to extract secondary performance information from the voltage difference message.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a block diagram of an implanted LVAD system;

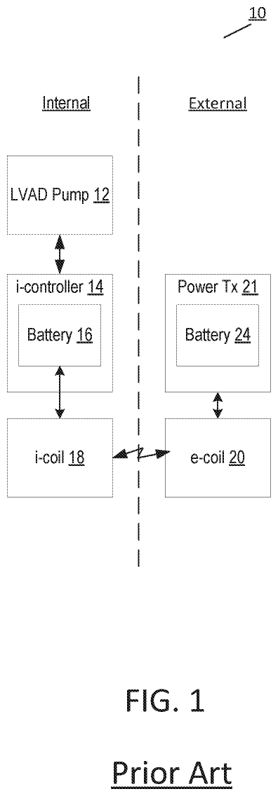

FIG. 2 is a block diagram of an embodiment of an LVAD system that implements a process of closed loop power regulation of a transcutaneous energy transfer system (TETS);

FIG. 3 is a block diagram of closed loop power regulation of a TETS that supplies power to an implanted medical device;

FIG. 4 is a flowchart of a process in an internal device of an implantable medical device for closed loop power regulation of a transcutaneous energy transfer system (TETS); and

FIG. 5 is a flowchart of a process in an external device of an implantable medical device for closed loop power regulation of a transcutaneous energy transfer system (TETS).

### DETAILED DESCRIPTION

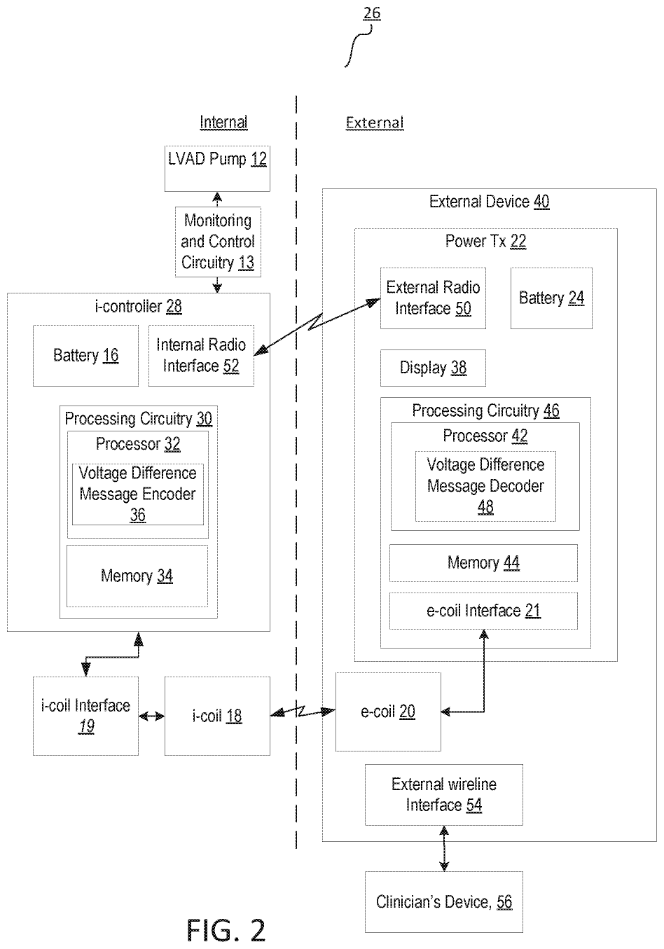

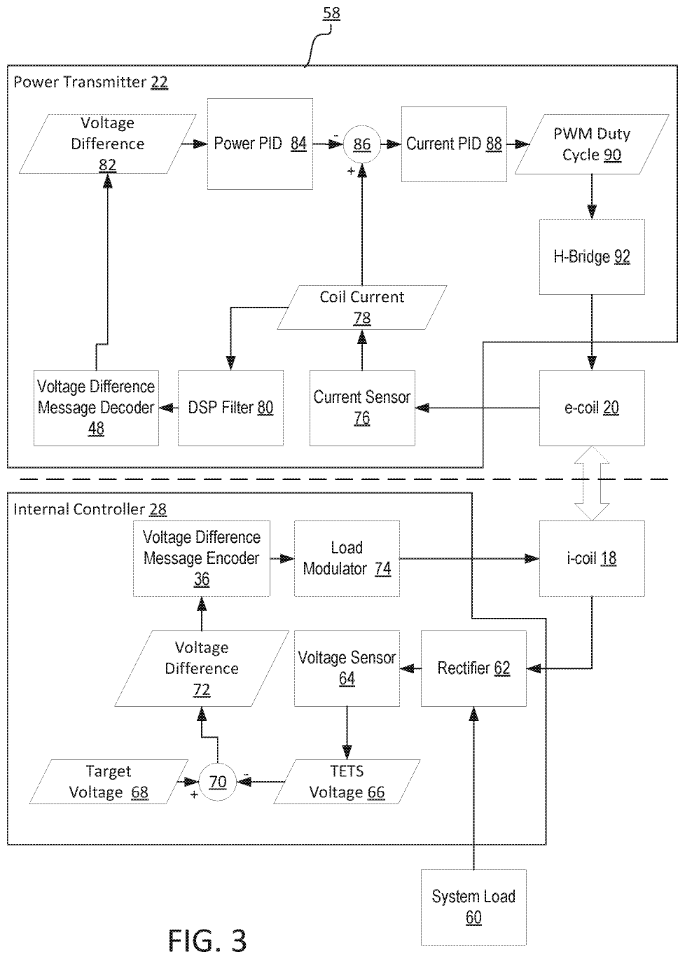

Some embodiments described herein are related to closed loop power regulation of a transcutaneous energy transfer system (TETS). FIG. 2 shows a block diagram of one example configuration of an implanted medical device system 26, which in some embodiments, is an LVAD system, having an internal component such as an internal controller (i-controller) 28 configured to perform functions described herein. The i-controller 28 may have processing circuitry 30 which may include a processor 32 and an internal memory 34. The processor 32 may be configured to execute computer instructions stored in the internal memory 34. Those instructions may include instructions to cause the processor to perform the processes described in more detail below. The processor 32 may therefore implement a voltage difference message encoder 36, which is described below with reference to FIG. 3. The internal memory 34 may be configured to also store an encoded voltage difference message encoded by the voltage difference message encoder 36.

An encoded voltage difference message may be transferred from the i-controller 28 to an external device 40, which may include a processor 42 and a memory 44 within processing circuitry 46, the external power transmitter 22 and the detachable battery 24, as well as the e-coil 20 in some embodiments. The memory 44 may be configured to store computer instructions to be executed by the processor 42 and may also be configured to store a decoded voltage difference message decoded by the voltage difference message decoder 48. The processor 42 may implement a voltage difference message decoder 48 which is described below with reference to FIG. 3. The external display 38 may be configured to display information received from the i-controller 28.

Electrical communication of signals and power between the internal components of i-controller 28 may be via communication busses and individual electrical conductors not shown in FIG. 2. For example, a multi-conductor address bus and data bus may connect processor 32 with internal memory 34. In some embodiments, an i-coil interface 19 associated with i-coil 18 may be included in the set of internal components making up the implanted medical device system 26. One purpose of i-coil interface 19 may be to modulate the alternating current applied to the i-coil 18 with signals from the i-controller 28 to be transmitted from the i-coil 18 to the e-coil 20 and/or to demodulate signals to be received by the i-coil 18 from the e-coil 20. In some embodiments, a purpose of the i-coil interface 19 is to provide conversion between the alternating current (AC) of the i-coil 18 and direct current (DC) to charge the internal battery 16. The power supplied to the i-coil 18 may be adjusted by varying the AC electrical current in the e-coil 20. Some or all functions of the i-coil interface 19 may be included in the i-controller 28 and/or the i-coil 18. Similarly, electrical communication of signals and power between the internal components of external device may be by communication busses and individual electrical conductors not shown in FIG. 2. For example, a multi-conductor address bus and data bus may connect processor 42 with memory 44. In some embodiments, an e-coil interface 21 associated with e-coil 20 may be included in the set of internal components making up the implanted medical device system 26. One purpose of e-coil interface 21 may be to modulate the alternating current applied to the e-coil 20 with signals from the processing circuitry 46 to be transmitted from the e-coil 20 to the i-coil 18 and/or to demodulate signals received by the e-coil 20 from the i-coil 18.

In some embodiments, the internal components of the implanted medical device system 26 may include monitoring and control circuitry 13. A purpose of monitoring and control circuitry 13 may include monitoring speed and temperature, for example, of the LVAD pump 12. Another purpose of the monitoring and control circuitry 13 may include controlling the speed of the LVAD pump 12. In some embodiments, some or all of the monitoring and control circuitry 13 may be incorporated into the LVAD pump 12 and/or the i-controller 28. In some embodiments, some or all of the functions performed by the monitoring and control circuitry 13 may be performed by the processing circuitry 30. Thus, in some embodiments, the monitoring and control circuitry 13 may include one or more temperature sensors embedded in the LVAD pump 12. Information obtained from and/or about the LVAD pump 12, such as speed and temperature, may be sent to the external device 40 to be displayed by external display 38.

The various internal components making up the LVAD system may be grouped into one or more separate housings. Similarly, the various external components making up the LVAD system may be grouped into one or more separate housings. Further, some of the components shown and described as being internal to the i-controller 28 may be instead, external to i-controller 28 in some embodiments. Similarly, some of the components shown and described as being internal to the external device 40 may be instead, external to external device 40 in some embodiments. Note further, the some of the functions performed by processor 32 may be performed instead by processor 42.

Note that transfer of information from the external device 40 to the internal memory 34, and vice versa, may be by wireless radio frequency (RF) transmission (over the air and through the body). Accordingly, in some embodiments, the external device 40 includes an external radio interface 50 and the i-controller 28 includes an internal radio interface 52. In some embodiments, the external radio interface 50 and the internal radio interface 52 are RF transceivers having both an RF receiver for receiving information wirelessly and an RF transmitter for transmitting information wirelessly. Such RF transceivers may be Bluetooth and/or Wi-Fi compliant, for example.

Also, information may be communicated to the i-controller 28 from the power transmitter 22 via the coils 18 and 20, by modulating a parameter of power transmission, such as modulating the frequency of the transmitted power, or by modulating a parameter of the i-coil interface 19, for example, by modulating a tuning capacitance of the i-coil interface 19 or by modulating the load level of the i-controller and/or the i-coil interface 19.

The external device 40 could be a patient's external device that has an external wireline interface 54 which provides an interface between the external device 40 and a clinician's device 56. The clinician's device might, for example, have a USB port and wireline interface 54 might include a USB port, so that a USB cable may connect the two ports. The clinician's device 56 may read data from the external device 40 and write information and control signaling to the external device 40, in some embodiments. In the alternative to a wireline connection, the wireline interface 54 could include or be replaced by a radio interface.

FIG. 3 is a block diagram of closed loop power regulation of a TETS that supplies power to an implanted medical device. The closed loop 58 includes components of the processing circuitry 30 of the i-controller 28 and components of the processing circuitry 45 of the power transmitter 22, as well as the internal coil 18 and external coil 20. The i-controller 28 includes the voltage difference message encoder 36 and the power transmitter 22 includes the voltage difference message decoder 48.

An object of the closed loop power regulation provided by the closed loop 58 is to apply a predetermined constant voltage to the system load 60 of the implanted medical device of the implanted medical device system 26. The system load 60 may be the load of the LVAD pump 12 or other implanted device, the load of the i-controller 28 (including the internal battery 16), and optionally also the load of the i-coil interface 19 and the monitoring and control circuitry 13. The system load 60 may be an electrical load at an input to a rectifier 62. The rectifier 62 also has an input that is electrically coupled to the i-coil 18. A purpose of the rectifier 62 is to rectify the voltage output of the i-coil 18. The rectification by the rectifier 62 may be based at least in part on the load presented by the system load 60. The rectified voltage output of the rectifier 62 is sensed by the voltage sensor 64. The voltage sensor 64 produces an output that is stored in the internal memory 34 of the i-controller 28 as the TETS voltage 66. The TETS voltage 66 is subtracted from a target voltage 68 by an adder 70 to produce the voltage difference 72. The target voltage 68 may be stored in the internal memory 34 of the i-controller 28 and may be equal to or be based on a desired voltage across the internal battery 16 of the i-controller 28. The voltage difference 72 is sent to the power transmitter 22 to be used to adjust the power transferred to the i-coil 18 in order to drive the voltage difference 72 to zero.

To send the voltage difference 72 to the power transmitter 22, the voltage difference 72 may first be encoded by the voltage difference message encoder 36 by, for example, modulating a digital form of the voltage difference 72 onto an analog signal. The encoded voltage difference message may be modulated onto the alternating current (AC) passing through the i-coil 18 by the load modulator 74. The load modulator 74 modulates the encoded voltage difference message onto the alternating current passing through the i-coil in such a way as to transmit the encoded voltage difference message to the power transmitter 22 via the mutual induction between the i-coil 18 and the e-coil 20. For example, the encoding and/or modulating by the voltage difference message encoder 36 and the load modulator 74 may include on off keying (OOK) and/or binary phase shift keying (BPSK) to encode and/or modulate the voltage difference message. Other modulation schemes may be employed such as multilevel amplitude shift keying (ASK) or higher order phase shift keying such as quadrature phase shift keying (QPSK). For example, in some embodiments, OOK may be used to signal the encoded voltage difference 72 and secondary performance information such as charging rate of the internal battery 16, power consumption by the LVAD pump 12, temperature of the internal electronics and/or the LVAD pump 12, as well as status of any of one or more processes implemented by the internal controller 28. In some embodiments, BPSK may be used to signal the encoded voltage difference 72 and secondary performance information.

In some embodiments, the secondary performance information may include anticipated performance changes as well as measured/past secondary performance information. For example, the power transmitter 22 may be instructed to initiate an internal battery 16 charging event. A battery charging event can cause a rapid change in power demand. The processing circuitry 46 may anticipate the change in power demand and alter the power transmission level, either directly or indirectly through changes in proportional integral derivative (PID) gains in order to better regulate the power level during the power demand change.

Thus, power is transferred from the power transmitter 22 to the internal controller 28 via the coils 18, 20 and, possibly simultaneously, the encoded voltage difference message is transferred from the internal controller 28 to the power transmitter 22 via the same two coils 18 and 22. The voltage difference message may include the voltage difference 72, as well as the secondary performance information as well as any other information to be sent with the voltage difference 72.

In the power transmitter 22, the current in the e-coil 20 is sensed by the current sensor 76 which outputs the e-coil current 78. The e-coil current 78 carries the encoded voltage difference message that was encoded by the voltage difference message encoder 36. The e-coil current 78 is filtered (and/or demodulated) by the digital signal processor (DSP) filter 80. For example, the DSP filter 80 could be a finite impulse response (FIR) filter. In some embodiments, the DSP filter 80 extracts the encoded voltage difference message that is carried by the e-coil current 78. The voltage difference message decoder 48 decodes the encoded voltage difference message to produce the decoded voltage difference 82. This may equal or approximately equal the voltage difference 72 determined as a difference between the TETS voltage 66 and the target voltage 68.

In some embodiments, there may be a delay between the time of determining a voltage difference 72 and the time of determining the corresponding voltage difference 82. This delay may affect how closely the closed loop 58 maintains the TETS voltage 66 at the target voltage 68 and may affect damping of the closed loop 58.

A power proportional integral derivative (PID) controller 84, in response to the voltage difference 82, generates a current adjustment signal that is subtracted from the coil current 78 by an adder 86 to produce current error signal. In response to the current error signal, a current PID controller 88 determines a pulse width modulation (PWM) duty cycle 90 to control the current in the e-coil 20 via a driver such as an H-bridge 92. A purpose of one or both of the power PID controller 84 and the current PID controller 88 may be to dampen any overshoot without excessive damping of the control loop.

An objective of the closed loop 58 is to continually drive the voltage difference 72, 82 toward zero so that the TETS voltage 66 is maintained at the target voltage 68. When the voltage difference 72, 82 is small, the current error signal input to the current PID controller 88 is small, resulting in a small change in the PWM duty cycle. When the change in the PWM duty cycle is small, the change in current driving the coil is small, which in turn results in only a small change in the TETS voltage 66. This small change in the TETS voltage 66 should result in even a smaller voltage difference 72. This causes the TETS voltage 66 to be maintained at or very close to the target voltage 68.

Note that in some embodiments, the communication between the i-controller 28 and the power transmitter 22 may be synchronized. In some embodiments, the AC signal applied to the e-coil 20 is used as the synchronization clock. This reduces complexity. A low quantity of communication pulses per data bit such as one communication pulse per data bit may be transferred over 4 to 8 cycles of the AC signal applied to the e-coil 20. This enables a fast enough update rate to drive the voltage difference to a negligible value. Note also that only the voltage difference 72 is fed back to the power transmitter, rather than the TETS voltage 66. This reduces the amount of data to be transmitted for closed loop power regulation.

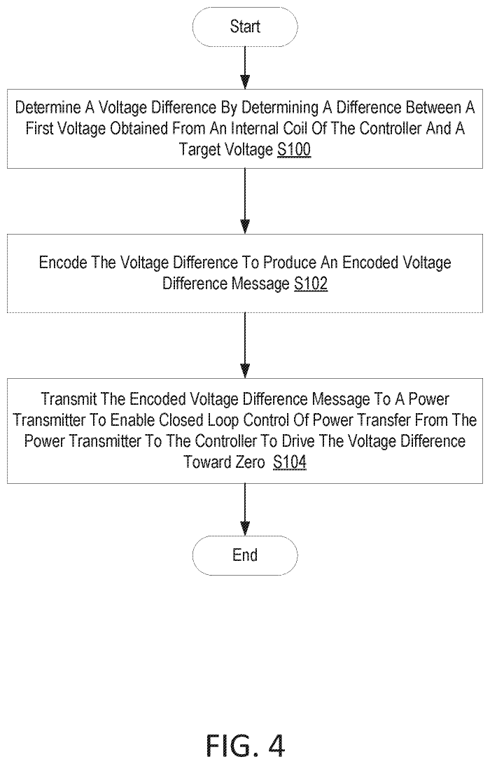

FIG. 4 is a flowchart of a process in an internal device such as i-controller 28 and internal coil 18 of an implanted medical device system 26 for closed loop power regulation of a transcutaneous energy transfer system (TETS). The process includes determining, via the processing circuitry 30, a voltage difference by determining a difference between a first voltage obtained from a supply voltage from rectifier 62 and the target voltage 68 (Block S100). The process also includes encoding the voltage difference 72 via the voltage difference message encoder 36 to produce an encoded voltage difference message (Block S102). The process further includes transmitting the encoded voltage difference message to a power transmitter 22 via the internal coil 18 to enable closed loop control of power transfer from the power transmitter 22 to the controller 28 to drive the voltage difference toward zero (Block S104).

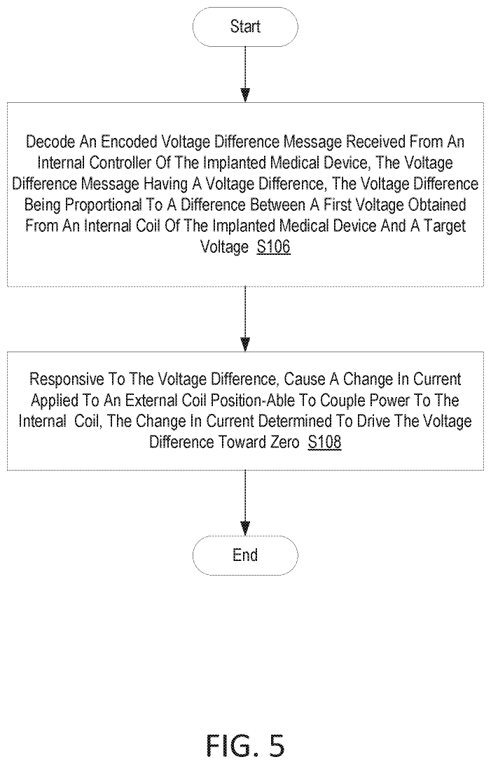

FIG. 5 is a flowchart of a process in an external device, such as a power transmitter 22, of an implanted medical device system 26 for closed loop power regulation of a transcutaneous energy transfer system (TETS). The process includes decoding, via the voltage difference message decoder 48, an encoded voltage difference message received from the internal controller 28 of the implanted medical device, the voltage difference message having a voltage difference 82, the voltage difference 82 being proportional to or equal to a difference between a first voltage 66 obtained from the internal coil 18 of the implanted medical device and a target voltage 68 (Block S106). The process further includes, responsive to the voltage difference 82, causing, via the processing circuitry 46, a change in current applied to an external coil 20 position-able to couple power to the internal coil 18, the change in current determined to drive the voltage difference 72 toward zero (Block S108).

Thus, according to one aspect, a controller 28 implantable within the body of a patient as part of an implanted medical device is provided. The controller 28 includes processing circuitry 30 configured to determine a voltage difference by determining a difference between a first voltage obtained from a rectified supply voltage of the controller and a target voltage, and encode the voltage difference to produce an encoded voltage difference message. The controller 28 also includes an internal coil 18 configured to transmit the encoded voltage difference message to a power transmitter 22 to enable closed loop control of power transfer from the power transmitter 22 to the controller to drive the voltage difference toward zero.

According to this aspect, in some embodiments, the encoding includes combining the voltage difference with secondary performance information. In some embodiments, the voltage difference and the secondary performance information are encoded using binary phase shift keying (BPSK). In some embodiments, the processing circuitry is further configured to synchronize transmission of the encoded voltage difference message to an alternating current (AC) applied to an external coil electromagnetically coupled to the internal coil. In some embodiments, the encoded voltage difference message modulates a load on the internal coil.

According to another aspect, a method implemented in a controller 28 implantable within the body of a patient as part of an implanted medical device is provided. The method includes determining a voltage difference by determining a difference between a first voltage obtained from an internal coil of the controller and a target voltage and encoding the voltage difference to produce an encoded voltage difference message. The method also includes transmitting the encoded voltage difference message to a power transmitter 22 via the internal coil 18 to enable closed loop control of power transfer from the power transmitter 22 to the controller 28 to drive the voltage difference toward zero.

According to this aspect, in some embodiments, the encoding includes combining the voltage difference with secondary performance information. In some embodiments, the voltage difference and the secondary performance information are encoded using binary phase shift keying (BPSK). In some embodiments, the transmission of the encoded voltage difference message is synchronized to an alternating current (AC) applied to an external coil electromagnetically coupled to the internal coil. In some embodiments, the encoded voltage difference message modulates a load on the internal coil.

According to yet another aspect, a power transmitter 22 configured to transmit power to an implanted medical device is provided. The power transmitter 22 includes processing circuitry 46 configured to decode an encoded voltage difference message received from an internal controller 28 of the implanted medical device, the encoded voltage difference message having a voltage difference, the voltage difference being proportional to a difference between a first voltage obtained from an internal coil 18 of the implanted medical device and a target voltage. The processing circuitry is further configured to, responsive to the voltage difference, cause a change in current applied to an external coil position-able to couple power to the internal coil 18, the change in current determined to drive the voltage difference toward zero.

According to this aspect, in some embodiments, causing the change in current applied to the external coil 20 includes adjusting a duty cycle of pulse width modulation of the current applied to the external coil 20 based at least in part on a difference between a current in the external coil and an adjustment signal based on the voltage difference. In some embodiments, the adjustment signal based at least in part on the voltage difference is generated by a power proportional integral derivative (PID) controller. In some embodiments, the processing circuitry further includes a digital filter to extract the encoded voltage difference message from a current signal sensed on the external coil. In some embodiments, the processing circuitry is further configured to extract secondary performance information from the encoded voltage difference message.

According to another embodiment, a method in a power transmitter configured to transmit power to an implanted medical device is provided. The method includes decoding an encoded voltage difference message received from an internal controller of the implanted medical device, the encoded voltage difference message having a voltage difference, the voltage difference being proportional to a difference between a first voltage obtained from a rectified supply voltage of the implanted medical device and a target voltage. The method further includes, responsive to the voltage difference, causing a change in current applied to an external coil position-able to couple power to an internal coil, the change in current determined to drive the voltage difference toward zero.

According to this aspect, in some embodiments, causing the change in current applied to the external coil includes adjusting a duty cycle of pulse width modulation of the current applied to the external coil based at least in part on a difference between a current in the external coil and an adjustment signal based at least in part on the voltage difference. In some embodiments, the adjustment signal based on the voltage difference is generated by a power proportional integral derivative (PID) controller. In some embodiments, the method further includes extracting by a digital filter, the encoded voltage difference message from a current signal sensed on the external coil. In some embodiments, the method further includes extracting secondary performance information from the encoded voltage difference message. In some embodiments, the voltage difference and the secondary performance information are encoded using binary phase shift keying (BPSK). In some embodiments, the transmission of the encoded voltage difference message is synchronized to an alternating current (AC) applied to an external coil electromagnetically coupled to the internal coil. In some embodiments, the encoded voltage difference message modulates a load on the internal coil.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media and memory may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope of the invention, which is limited only by the following claims.

What is claimed is:

1. A controller implantable within the body of a patient as part of an implanted medical device, the controller comprising:
processing circuitry configured to:
determine a voltage difference by determining a difference between a first voltage obtained from a rectified supply voltage of the controller and a target voltage; and
encode the voltage difference to produce an encoded voltage difference message; and
an internal coil configured to transmit the encoded voltage difference message to a power transmitter to enable closed loop control of power transfer from the power transmitter to the controller to drive the voltage difference toward zero.

2. The controller of claim 1, wherein the encoding includes combining the voltage difference with secondary performance information.

3. The controller of claim 1, wherein the voltage difference and the secondary performance information are encoded using binary phase shift keying (BPSK).

4. The controller of claim 1, wherein the processing circuitry is further configured to synchronize transmission of the encoded voltage difference message to an alternating current (AC) applied to an external coil electromagnetically coupled to the internal coil.

5. The controller of claim 1, wherein the encoded voltage difference message modulates a load on the internal coil.

6. A method implemented in a controller implantable within the body of a patient as part of an implanted medical device, the method including:
determining a voltage difference by determining a difference between a first voltage obtained from an internal coil of the controller and a target voltage;
encoding the voltage difference to produce an encoded voltage difference message; and
transmitting the encoded voltage difference message to a power transmitter via the internal coil to enable closed loop control of power transfer from the power transmitter to the controller to drive the voltage difference toward zero.

7. The method of claim 6, wherein the encoding includes combining the voltage difference with secondary performance information.

8. The method of claim 6, wherein the voltage difference and the secondary performance information are encoded using binary phase shift keying (BPSK).

9. The method of claim 6, wherein the transmission of the encoded voltage difference message is synchronized to an alternating current (AC) applied to an external coil electromagnetically coupled to the internal coil.

10. The method of claim 6, wherein the encoded voltage difference message modulates a load on the internal coil.

11. A power transmitter configured to transmit power to an implanted medical device, the power transmitter comprising:
processing circuitry configured to:
decode an encoded voltage difference message received from an internal controller of the implanted medical device, the encoded voltage difference message having a voltage difference, the voltage difference being proportional to a difference between a first voltage obtained from an internal coil of the implanted medical device and a target voltage; and
responsive to the voltage difference, cause a change in current applied to an external coil position-able to couple power to the internal coil, the change in current determined to drive the voltage difference toward zero.

12. The power transmitter of claim 11, wherein causing the change in current applied to the external coil includes adjusting a duty cycle of pulse width modulation of the current applied to the external coil based at least in part on a difference between a current in the external coil and an adjustment signal based on the voltage difference.

13. The power transmitter of claim 12, wherein the adjustment signal based at least in part on the voltage difference is generated by a power proportional integral derivative (PID) controller.

14. The power transmitter of claim 11, wherein the processing circuitry further includes a digital filter to extract the encoded voltage difference message from a current signal sensed on the external coil.

15. The power transmitter of claim 11, wherein the processing circuitry is further configured to extract secondary performance information from the encoded voltage difference message.

16. The power transmitter of claim 11, wherein the voltage difference and the secondary performance information are encoded using binary phase shift keying (BPSK).

17. The power transmitter of claim 11, wherein the processing circuitry is further configured to synchronize transmission of the encoded voltage difference message to an alternating current (AC) applied to an external coil electromagnetically coupled to the internal coil.

18. The power transmitter of claim 11, wherein the encoded voltage difference message modulates a load on the internal coil.
